(19) United States
(12) Patent Application Publication
(10) Pub. No.: US 2024/0286995 A1
(43) Pub. Date: Aug. 29, 2024

(54) BIO-BASED ISETHIONATE COMPOUNDS

(71) Applicant: Clariant International Ltd, Muttenz (CH)

(72) Inventors: Catherine BREFFA, Frankfurt am Main (DE); Carsten COHRS, Frankfurt am Main (DE)

(73) Assignee: Clariant International Ltd, Muttenz (CH)

(21) Appl. No.: 18/573,548

(22) PCT Filed: Jun. 24, 2022
(86) PCT No.: PCT/EP2022/067407

§ 371 Date: Dec. 22, 2023

(30) Foreign Application Priority Data
Jun. 30, 2021 (EP) 21183003.9
Jul. 6, 2021 (EP) 21184125.9

(51) Int. Cl. C07C 309/08; A61K 8/46; C07C 309/12; C11D 1/14; C11D 1/28

(52) U.S. Cl. CPC C07C 309/08; A61K 8/466; C07C 309/12; C11D 1/14; C11D 1/28; A61K 2800/10

(57) ABSTRACT

Disclosed herein is a compound of Formula (I), wherein Q⁺ is a cation.

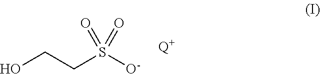

The compound of Formula (I) has at least 50 wt-% bio-based carbon content, relative to the total mass of carbon in the compound.

The invention relates to bio-based isethionate compounds, their preparation, their uses, and formulations comprising the bio-based isethionate compounds.

Many ingredients for use in cosmetic or household cleaning formulations are traditionally derived from crude oil. Environmental, economic and sustainability questions are restricting the use of products derived from this limited resource: synthetic surfactants, for example, have been blamed for environmental incidents, particularly vis-à-vis aquatic problems in rivers and lakes. Therefore, there is a desire to identify more sustainable and biodegradable, yet gentle and effective materials. Indeed, consumers are very interested in “natural” products including products with a high percentage of “natural” compounds and/or compounds that are derived from renewable materials. Consumers perceive compounds derived from natural materials to be gentler and more environmentally friendly.

Nevertheless, the availability of renewable ingredients for use in cosmetic or household cleaning formulations is still limited. There is a need for ingredients including surfactants that are not only renewable, but also provide excellent performance. There is a need for cosmetic or household cleaning ingredients including surfactants that can provide the excellent performance of modern ingredients yet are from more renewable sources.

The present invention relates to a compound of Formula (I)

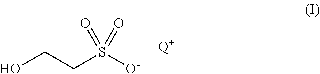

wherein Q⁺ is a cation; and
wherein the compound of Formula (I) has at least 50 wt-%, preferably at least 80 wt-%, particularly preferably 100 wt-%, bio-based carbon content, relative to the total mass of carbon in the compound.

The present invention also relates to a compound of Formula (II)

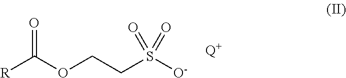

wherein Q⁺ is a cation; and
wherein R is selected from saturated hydrocarbon chains having 5 to 23 carbon atoms, unsaturated hydrocarbon chains having 5 to 23 carbon atoms, and combinations thereof, or the R—C═O residue in Formula (II) is derived from coconut fatty acids; and
wherein the compound of Formula (II) has at least 50 wt-%, preferably at least 80 wt-%, particularly preferably 100 wt-%, bio-based carbon content, relative to the total mass of carbon in the compound.

In preferred embodiments, the bio-based carbon content as used herein is measured according to standard ASTM D6866-12, Method B. In preferred embodiments, the bio-based carbon content of the compound of Formula (I) as used herein is measured according to standard ASTM D6866-12, Method B. In preferred embodiments, the bio-based carbon content of the compound of Formula (II) as used herein is measured according to standard ASTM D6866-12, Method B. In preferred embodiments, the bio-based carbon content of the compound of Formula (III) as used herein is measured according to standard ASTM D6866-12, Method B. In preferred embodiments, the bio-based carbon content of ethylene oxide as used herein is measured according to standard ASTM D6866-12, Method B. In preferred embodiments, the bio-based carbon content of ethylene as used herein is measured according to standard ASTM D6866-12. Method B.

The “bio-based content” is reported in ASTM D6866-12, Method B (see section 3.3.9 of ASTM D6866-12). “Bio-based carbon content”, “bio-based content”. “biogenic carbon content”, “bio-based content”, “biomass-derived carbon” herein refer to the same thing and are all measured in wt-%. Herein, the term ‘bio-based carbon content’ is used. ASTM D6866-12, Method B lab results report the percentage of bio-based carbon content relative to total carbon, and not to total mass of the sample or molecular weight. A comment on bio-based carbon content calculation: ASTM D6866-12, Method B (see section 9 of ASTM D6866-12) requires the percent modern carbon value (pMC) reported to be multiplied by a correction factor of 0.95 to account for excess carbon-14 in the atmosphere due to nuclear weapons testing. Hence the term “bio-based carbon content” as used herein (if measured according to standard ASTM D6866-12, Method B) is defined by the equation: Bio-based carbon content=pMC*0.95(%)

In preferred embodiments, the bio-based carbon content as used herein is measured according to standard ASTM D6866-21, Method B. In preferred embodiments, the bio-based carbon content of the compound of Formula (I) as used herein is measured according to standard ASTM D6866-21, Method B. In preferred embodiments, the bio-based carbon content of the compound of Formula (II) as used herein is measured according to standard ASTM D6866-21, Method B. In preferred embodiments, the bio-based carbon content of the compound of Formula (III) as used herein is measured according to standard ASTM D6866-21, Method B. In preferred embodiments, the bio-based carbon content of ethylene oxide as used herein is measured according to standard ASTM D6866-21, Method B. In preferred embodiments, the bio-based carbon content of ethylene as used herein is measured according to standard ASTM D6866-21, Method B.

The “bio-based content” is reported in ASTM D6866-21, Method B. “Bio-based carbon content”, “bio-based content”, “biogenic carbon content”, “bio-based content”, “biomass-derived carbon” herein refer to the same thing and are all measured in wt-%. Herein, the term ‘bio-based carbon content’ is used. ASTM D6866-21, Method B lab results report the percentage of bio-based carbon content relative to total carbon, and not to total mass of the sample or molecular weight.

A review on measurement methods of bio-based carbon content for biomass-based chemicals and plastics is given by Massao Kunioka in Radioisotopes, 62, 901-925 (2013).

Bio-based products are part of the natural carbon cycle. If these products are incinerated or biodegraded, the quantity of carbon dioxide that is emitted corresponds to the quantity fixed by photosynthesis during biomass growth.

Bio-based compounds of Formulae (I) and (II) can, for example, be prepared from bio-based ethylene oxide, which can in turn, for example, be prepared from bio-based ethylene. Bio-based ethylene can, for example, be obtained from bio-based ethanol by dehydration. Bio-based ethanol can, for example, be obtained from sugar by fermentation.

In preferred embodiments, the compound of Formula (I) has at least 60 wt-%, preferably at least 70 wt-%, preferably at least 75 wt-%, preferably at least 80 wt-%, preferably at least 85 wt-%, preferably at least 90 wt-%, preferably at least 95 wt-%, particularly preferably 100 wt-%, bio-based carbon content, relative to the total mass of carbon in the compound.

In preferred embodiments, the compound of Formula (I) is derived from ethylene oxide, wherein the ethylene oxide has at least 50 wt-%, preferably at least 60 wt-%, preferably at least 70 wt-%, preferably at least 75 wt-%, preferably at least 80 wt-%, preferably at least 85 wt-%, preferably at least 90 wt-%, preferably at least 95 wt-%, particularly preferably 100 wt-%, bio-based carbon content, relative to the total mass of carbon in ethylene oxide.

Preferably, said ethylene oxide is derived from ethylene, wherein the ethylene has at least 50 wt-%, preferably at least 60 wt-%, preferably at least 70 wt-%, preferably at least 75 wt-%, preferably at least 80 wt-%, preferably at least 85 wt-%, preferably at least 90 wt-%, preferably at least 95 wt-%, particularly preferably 100 wt-%, bio-based carbon content, relative to the total mass of carbon in ethylene.

Preferred compounds of Formula (I) are compounds of Formula (I) wherein Q⁺ is selected from H⁺, NH₄⁺, Li⁺, Na⁺, K⁺, ½ Ca⁺⁺, ½ Mg⁺⁺, ½ Zn⁺⁺, ⅓ Al⁺⁺⁺, and combinations thereof. More preferred compounds of Formula (I) are compounds of Formula (I) wherein Q⁺ is selected from H⁺, NH₄⁺, Na⁺, K⁺, and combinations thereof. Even more preferred compounds of Formula (I) are compounds of Formula (I) wherein Q⁺ is selected from Na⁺, K⁺, and combinations thereof. A particularly preferred compound of Formula (I) is a compound of Formula (I) wherein Q⁺ is Na⁺. A particularly preferred compound of Formula (I) is sodium isethionate. Another preferred compound of Formula (I) is potassium isethionate.

In preferred embodiments, the compound of Formula (II) has at least 60 wt-%, preferably at least 70 wt-%, preferably at least 75 wt-%, preferably at least 80 wt-10%, preferably at least 85 wt-%, preferably at least 90 wt-%, preferably at least 95 wt-%, particularly preferably 100 wt-%, bio-based carbon content, relative to the total mass of carbon in the compound.

In preferred embodiments, the compound of Formula (II) is derived from ethylene oxide, wherein the ethylene oxide has at least 50 wt-%, preferably at least 60 wt-%, preferably at least 70 wt-%, preferably at least 75 wt-%, preferably at least 80 wt-%, preferably at least 85 wt-%, preferably at least 90 wt-%, preferably at least 95 wt-%, particularly preferably 100 wt-%, bio-based carbon content, relative to the total mass of carbon in ethylene oxide.

Preferably, said ethylene oxide is derived from ethylene, wherein the ethylene has at least 50 wt-%, preferably at least 60 wt-%, preferably at least 70 wt-%, preferably at least 75 wt-%, preferably at least 80 wt-%, preferably at least 85 wt-%, preferably at least 90 wt-%, preferably at least 95 wt-%, particularly preferably 100 wt-%, bio-based carbon content.

Preferred compounds of Formula (II) are compounds of Formula (II) wherein Q⁺ is selected from H⁺, NH₄⁺, Li⁺, Na⁺, K⁺, ½ Ca⁺⁺, ½ Mg⁺⁺, ½ Zn⁺⁺, ⅓ Al⁺⁺⁺, and combinations thereof. More preferred compounds of Formula (II) are compounds of Formula (II) wherein Q⁺ is selected from H⁺, NH₄⁺, Na⁺, K⁺, and combinations thereof. Even more preferred compounds of Formula (II) are compounds of Formula (II) wherein Q⁺ is selected from NH₄⁺, Na⁺, and combinations thereof. Particularly preferred compounds of Formula (II) are compounds of Formula (II) wherein Q⁺ is Na⁺. Also preferred compounds of Formula (II) are compounds of Formula (II) wherein Q⁺ is NH₄⁺.

Preferred compounds of Formula (II) are compounds of Formula (II) wherein R is selected from saturated hydrocarbon chains having 7 to 17 carbon atoms, or the R—C═O residue in Formula (II) is derived from coconut fatty acids.

More preferred compounds of Formula (II) are compounds of Formula (II) wherein R is selected from saturated hydrocarbon chains having 7 to 17 carbon atoms.

Even more preferred compounds of Formula (II) are compounds of Formula (II) wherein R is selected from saturated hydrocarbon chains having 9 to 13 carbon atoms,

Particularly preferred compounds of Formula (II) are compounds of Formula (II) wherein R is a saturated hydrocarbon chain having 11 carbon atoms,

Also particularly preferred compounds of Formula (II) are compounds of Formula (II) wherein the R—C═O residue in Formula (II) is derived from coconut fatty acids.

As used herein, the expression “the R—C═O residue in Formula (II) is derived from coconut fatty acids” preferably means that the carbon chain length distribution in the R—C═O residue in Formula (II) corresponds to the carbon chain length distribution of the fatty acids (e.g. bound as triglycerides) in coconut oil.

Particularly preferred compounds of Formula (II) are sodium lauroyl isethionate or sodium cocoyl isethionate. A particularly preferred compound of Formula (II) is sodium lauroyl isethionate. Another particularly preferred compound of Formula (II) is sodium cocoyl isethionate.

Sodium cocoyl isethionate is also known as coconut fatty acid isethionate-sodium salt. Sodium cocoyl isethionate can, for example, be in the form of pellets, flakes, chips, granules, or a powder.

Also preferred compounds of Formula (II) are ammonium lauroyl isethionate or ammonium cocoyl isethionate.

In another preferred embodiment, R in Formula (II) is selected from saturated hydrocarbon chains having 7 to 9 carbon atoms,

In another preferred embodiment, R in Formula (II) is selected from saturated hydrocarbon chains having 11 to 13 carbon atoms,

In another preferred embodiment, R in Formula (II) is selected from saturated hydrocarbon chains having 15 to 17 carbon atoms,

In another preferred embodiment, R in Formula (II) is selected from unsaturated hydrocarbon chains having 7 to 17 carbon atoms. Preferably, the unsaturated hydrocarbon chain is a hydrocarbon chain having one, two, three or more double bonds, preferably one, two or three double bonds.

Further examples of preferred compounds of Formula (II) are sodium capryloyl isethionate, sodium caproyl isethionate, sodium myristoyl isethionate, sodium palmitoyl isethionate, sodium stearoyl isethionate, sodium oleoyl isethionate, ammonium capryloyl isethionate, ammonium caproyl isethionate, ammonium myristoyl isethionate, ammonium palmitoyl isethionate, ammonium stearoyl isethionate, ammonium oleoyl isethionate.

The present invention also relates to a process for preparing a compound of Formula (I)

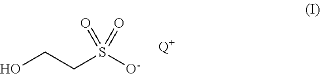

wherein Q⁺ is a cation; and
wherein the compound of Formula (I) has at least 50 wt-%, preferably at least 80 wt-%, particularly preferably 100 wt-%, bio-based carbon content, relative to the total mass of carbon in the compound;
wherein the process comprises reacting ethylene oxide with a bisulfite,
wherein the ethylene oxide has at least 50 wt-%, preferably at least 80 wt-%, particularly preferably 100 wt-%, bio-based carbon content, relative to the total mass of carbon in ethylene oxide.

Preferred compounds of Formula (I) are described further above.

Suitable bisulfites are known to a person skilled in the art.

The bisulfite can be used in any form. The bisulfite may, for example, be used as a solid or as an aqueous solution. The bisulfite may, for example, be prepared in situ. In one embodiment, the bisulfite is prepared in situ from sulfur dioxide and a base, preferably a hydroxide, carbonate and/or bicarbonate.

In a preferred embodiment, the bisulfite is sodium bisulfite. Sodium bisulfite is also known as sodium hydrogen sulfite or NaHSO₃.

Sodium bisulfite can be used in any form. Sodium bisulfite may, for example, be used as a solid or as an aqueous solution. Sodium bisulfite may, for example, be prepared in situ. In one embodiment, sodium bisulfite is prepared in situ from sulfur dioxide and a base, preferably sodium hydroxide, sodium carbonate and/or sodium bicarbonate.

In a preferred embodiment, the bisulfite is sodium bisulfite, and Q⁺ in Formula (I) is Na⁺. In a preferred embodiment, the bisulfite is sodium bisulfite, and the compound of Formula (I) is sodium isethionate.

Typically, the reaction of ethylene oxide with a bisulfite is carried out in the presence of a solvent. Suitable solvents are known to a person skilled in the art. A particularly preferred solvent is water. The pH may be adjusted as needed. In some embodiments, the reaction is carried out under inert atmosphere, for example nitrogen atmosphere or argon atmosphere.

The reaction of ethylene oxide with a bisulfite may, for example, be carried out at a temperature of from 20 to 100° C., preferably from 60 to 90° C., more preferably from 70 to 80° C.

In a preferred embodiment, the compound of Formula (I) of the present invention is obtained by the process of the present invention for preparing a compound of Formula (I).

The present invention also relates to a process for preparing a compound of Formula (II)

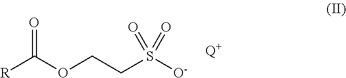

wherein Q⁺ is a cation; and
wherein R is selected from saturated hydrocarbon chains having 5 to 23 carbon atoms, unsaturated hydrocarbon chains having 5 to 23 carbon atoms, and combinations thereof, or the R—C═O residue in Formula (II) is derived from coconut fatty acids; and
wherein the compound of Formula (II) has at least 50 wt-%, preferably at least 80 wt-%, particularly preferably 100 wt-%, bio-based carbon content, relative to the total mass of carbon in the compound;
wherein the process comprises reacting a compound of Formula (I)

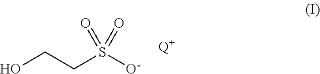

wherein Q⁺ is a cation; and
wherein the compound of Formula (I) has at least 50 wt-%, preferably at least 80 wt-%, particularly preferably 100 wt-%, bio-based carbon content, relative to the total mass of carbon in the compound;
with a compound of Formula (III)

RC(═O)OH (III)
wherein R is as defined in Formula (II).

Preferred compounds of Formula (II) are described further above.

In a preferred embodiment, the reaction of a compound of Formula (I) with a compound of Formula (III) is carried out in the presence of a catalyst. Suitable catalysts are known to a person skilled in the art.

The reaction of a compound of Formula (I) with a compound of Formula (III) may be carried out in the absence or in the presence of a solvent. In one embodiment, said reaction is carried out in the absence of a solvent. In another embodiment, said reaction is carried out in the presence of a solvent. Suitable solvents are known to a person skilled in the art.

The reaction of a compound of Formula (I) with a compound of Formula (III) may, for example, be carried out at a temperature of from 100 to 250° C., preferably from 180 to 240° C., more preferably from 200 to 230° C.

In preferred embodiments, the compound of Formula (III) has at least 50 wt-%, preferably at least 60 wt-%, preferably at least 70 wt-%, preferably at least 75 wt-%, preferably at least 80 wt-%, preferably at least 85 wt-%, preferably at least 90 wt-%, preferably at least 95 wt-%, particularly preferably 100 wt-%, bio-based carbon content, relative to the total mass of carbon in the compound.

In a preferred embodiment, the compound of Formula (II) of the present invention is obtained by the process of the present invention for preparing a compound of Formula (II).

Compounds of Formula (I) are versatile bio-based precursors for the preparation of bio-based compounds. For example, compounds of Formula (I) are useful as precursors for the preparation of compounds of Formula (II).

The present invention also relates to the use of a compound of Formula (I) as defined herein as a precursor for the preparation of a compound of Formula (II) as defined herein.

In a preferred embodiment, sodium isethionate is used as a precursor for the preparation of sodium lauroyl isethionate or sodium cocoyl isethionate. In a particularly preferred embodiment, sodium isethionate is used as a precursor for the preparation of sodium lauroyl isethionate. In another particularly preferred embodiment, sodium isethionate is used as a precursor for the preparation of sodium cocoyl isethionate.

The present invention also relates to the use of a compound of Formula (I) as defined herein or a compound of Formula (II) as defined herein in a cosmetic formulation or a household cleaning formulation.

Advantageously, the compounds of Formulae (I) and (II) show good skin compatibility and skin feel.

Advantageously, the compounds of Formula (II) show good foam formation, foam stability and clean(s)ing properties.

In a preferred embodiment, the formulation is selected from the group consisting of soap bars and liquid cleansers. The term “soap bar” or “bar soap” as used herein encompasses syndet bars and combo bars. In a preferred embodiment, the formulation is selected from the group consisting of syndet bars, combo bars, and liquid cleansers. In a particularly preferred embodiment, the formulation is a syndet bar or a combo bar. In a syndet bar, no conventional soap is used. In a combo bar, soap and detergent are used together. In a particularly preferred embodiment, the formulation is a syndet bar. In a particularly preferred embodiment, the formulation is a combo bar. In a particularly preferred embodiment, the formulation is a liquid cleanser.

In a preferred embodiment, the cosmetic composition is a solid cleansing formulation or a liquid cleansing formulation. Preferably, said formulations are for cleansing hair and/or skin. In a particular embodiment, the solid cleansing formulation is SLES-free. In a particular embodiment, the liquid cleansing formulation is SLES-free. “SLES” as used herein refers to sodium lauryl ether sulfate.

In at least one embodiment, the formulation is a cosmetic formulation for cleansing hair and/or skin.

In a preferred embodiment, the household cleaning formulation is a hand dishwashing formulation or a hard surface cleaner.

In a particularly preferred embodiment, the compound of Formula (II) is used as a surfactant.

The present invention also relates to the use of a compound of Formula (II) as defined herein as a surfactant.

The present invention also relates to a formulation comprising
(a) from 0.1 to 90 wt-% of a compound of Formula (I) as defined herein or a compound of Formula (II) as defined herein; and
(b) from 10 to 99.9 wt-% of one or more further components.

In at least one embodiment, the formulation is selected from the group consisting of cosmetic formulations and household cleaning formulations.

In at least one embodiment, the formulation is selected from the group consisting of shampoo, body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, soap bar, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock.

In at least one embodiment, the formulation is a formulation selected from the group consisting of shampoo, body wash, facial cleanser, cleansing mask, bubble bath, bath oil, cleansing milk, micellar water, make-up remover, cleansing wipes, perfume, soaps, soap bars, shaving soaps, shaving foams, cleansing foams, and hair mousse.

In a preferred embodiment, the formulation is selected from the group consisting of soap bars and liquid cleansers. The term “soap bar” or “bar soap” as used herein encompasses syndet bars and combo bars. In a preferred embodiment, the formulation is selected from the group consisting of syndet bars, combo bars, and liquid cleansers. In a particularly preferred embodiment, the formulation is a syndet bar or a combo bar. In a syndet bar, no conventional soap is used. In a combo bar, soap and detergent are used together. In a particularly preferred embodiment, the formulation is a syndet bar. In a particularly preferred embodiment, the formulation is a combo bar. In a particularly preferred embodiment, the formulation is a liquid cleanser.

In a preferred embodiment, the cosmetic composition is a solid cleansing formulation or a liquid cleansing formulation. Preferably, said formulations are for cleansing hair and/or skin. In a particular embodiment, the solid cleansing formulation is SLES-free. In a particular embodiment, the liquid cleansing formulation is SLES-free. “SLES” as used herein refers to sodium lauryl ether sulfate.

In at least one embodiment, the formulation is a cosmetic formulation for cleansing hair and/or skin.

In a preferred embodiment, the household cleaning formulation is a hand dishwashing formulation or a hard surface cleaner.

The formulation of the present invention comprises
(a) from 0.1 to 90 wt-% of a compound of Formula (I) as defined herein or a compound of Formula (II) as defined herein; and
(b) from 10 to 99.9 wt-% of one or more further components.

In a preferred embodiment, the formulation comprises
(a) from 1 to 85 wt-%, preferably from 1 to 70 wt-%, more preferably from 1 to 60 wt-%, more preferably from 1 to 50 wt-%, of a compound of Formula (I) as defined herein or a compound of Formula (II) as defined herein; and
(b) from 15 to 99 wt-%, preferably from 30 to 99 wt-%, more preferably from 40 to 99 wt-%, more preferably from 50 to 99 wt-%, of one or more further components.

In a preferred embodiment, the formulation comprises
(a) from 1 to 15 wt-%, preferably from 1 to 10 wt-%, more preferably from 2 to 10 wt-%, more preferably from 2 to 5 wt-%, of a compound of Formula (I) as defined herein; and
(b) from 85 to 99 wt-%, preferably from 90 to 99 wt-%, more preferably from 90 to 98 wt-%, more preferably from 95 to 98 wt-%, of one or more further components.

In a preferred embodiment, the formulation is a solid cosmetic formulation (e.g. a syndet bar or a combo bar) and comprises
(a) from 1 to 85 wt-%, preferably from 5 to 70 wt-%, more preferably from 15 to 60 wt-%, more preferably from 20 to 50 wt-%, of a compound of Formula (II) as defined herein; and
(b) from 15 to 99 wt-%, preferably from 30 to 95 wt-%, more preferably from 40 to 85 wt-%, more preferably from 50 to 80 wt-%, of one or more further components.

In a preferred embodiment, the formulation is a liquid cosmetic formulation (e.g. a liquid cleansing formulation, also referred to as liquid cleanser) and comprises
(a) from 1 to 25 wt-%, preferably from 1 to 20 wt-%, more preferably from 2 to 15 wt-%, more preferably from 2 to 10 wt-%, of a compound of Formula (II) as defined herein; and
(b) from 75 to 99 wt-%, preferably from 80 to 99 wt-%, more preferably from 85 to 98 wt-%, more preferably from 90 to 98 wt-%, of one or more further components.

In a preferred embodiment, the formulation is a household cleaning formulation and comprises
(a) from 1 to 50 wt-%, preferably from 2 to 40 wt-%, more preferably from 3 to 30 wt-%, more preferably from 5 to 20 wt-%, of a compound of Formula (II) as defined herein; and
(b) from 50 to 99 wt-%, preferably from 60 to 98 wt-%, more preferably from 70 to 97 wt-%, more preferably from 80 to 95 wt-%, of one or more further components.

In at least one embodiment, the formulation is an aqueous solution.

In at least one embodiment, the formulation comprises solvent. In at least one embodiment, the formulation comprises a solvent, wherein the solvent comprises water and/or alcohol. Solvent is useful for providing the compounds used in present invention in liquid form. In at least one embodiment, the solvent is cosmetically acceptable. In at least one embodiment, the formulation comprises at least 10 wt.-%, preferably at least 20 wt.-%, more preferably at least 30 wt.-%, even more preferably at least 50 wt.-% water. Water is useful for economic reasons but also because it is cosmetically acceptable. Optionally, the formulation comprises water-miscible or water-soluble solvents, such as lower alkyl alcohols. In at least one embodiment, the formulation comprises C1-C5 alkyl monohydric alcohols, preferably C2-C3 alkyl monohydric alcohols. The alcohols which may be present are in particular lower monohydric or polyhydric alcohols having 1 to 4 carbon atoms customarily used for cosmetic purposes, such as preferably ethanol and isopropanol.

Optionally, the formulation comprises a water-soluble polyhydric alcohol. In at least one embodiment, the water-soluble polyhydric alcohols are polyhydric alcohols having two or more hydroxyl groups in the molecule. In at least one embodiment, the water-soluble polyhydric alcohol is selected from the group consisting of: dihydric alcohols such as ethylene glycol, propylene glycol, trimethylene glycol, 1,2-butylene glycol, 1,3-butylene glycol, 1,4-butylene glycol, tetramethylene glycol, 2,3-butylene glycol, pentamethylene glycol, 2-butene-1,4-diol, hexylene glycol, octylene glycol; trihydric alcohols such as glycerine, trimethylol propane, 1,2,6-hexanetriol and the like; tetrahydric alcohols such as pentaerythritol; pentahydric alcohols such as xylytol; hexahydric alcohols such as sorbitol, mannitol; polyhydric alcohol polymers such as diethylene glycol, dipropylene glycol, polyethylene glycol, polypropylene glycol, tetraethylene glycol, diglycerine, polyethylene glycol, triglycerine, tetraglycerine, polyglycerine; dihydric alcohol alkyl ethers such as ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, ethylene glycol isopropyl ether, ethylene glycol dimethyl ether, ethylene glycol diethyl ether, ethylene glycol dibutyl ether; dihydric alcohol alkyl ethers such as diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methyl ethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether; dihydric alcohol ether esters such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate; glycerine monoalkyl ethers such as xyl alcohol, selachyl alcohol, batyl alcohol: sugar alcohols such as sorbitol, maltitol, maltotriose, mannitol, sucrose, erythritol, glucose, fructose, starch sugar, maltose, xylytose, starch sugar reduced alcohol, glysolid, tetrahydrofurfuryl alcohol, POE tetrahydrofurfuryl alcohol, POP butyl ether, POP POE butyl ether, tripolyoxypropylene glycerine ether, POP glycerine ether, POP glycerine ether phosphoric acid, POP POE pentaerythritol ether, and mixtures thereof.

In a preferred embodiment, the formulation comprises a solvent selected from the group consisting of water, glycols, ethanol, and mixtures thereof. In a preferred embodiment, the formulation comprises water.

In a preferred embodiment, the formulation comprises an aqueous, alcoholic or aqueous-alcoholic solvent, wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or mixtures thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol. 1,2-propylene glycol and 1,3-propylene glycol.

Natural solvents can also be used. In at least one embodiment, the formulation comprises a solvent selected from the group consisting of plant oil, honey, plant-derived sugar compositions, and mixtures thereof.

In at least one embodiment, the formulation comprises additives common in cosmetology, pharmacy, and dermatology, which are hereinafter called auxiliaries. In at least one embodiment, the auxiliary is selected from the group consisting of oily substances, emulsifiers, coemulsifiers, cationic polymers, film formers, superfatting agents, stabilizers, active biogenic substances, glycerol, preservatives, pearlizing agents, dyes and fragrances, solvents, opacifiers, functional acids, and also protein derivatives such as gelatin, collagen hydrolysates, natural or synthetic-based polypeptides, egg yolk lecithin, lanolin and lanolin derivatives, fatty alcohols, silicones, deodorants, substances with a keratolytic and keratoplastic action, enzymes, and/or carriers/solvents.

In at least one embodiment, the formulation comprises water soluble vitamins and their derivatives, water soluble amino acids and their salts and/or derivatives, viscosity modifiers, dyes, nonvolatile solvents or diluents (water soluble and insoluble), pearlescent aids, thickeners, foam boosters, additional surfactants or nonionic co-surfactants, pediculocides, pH adjusting agents, perfumes, preservatives, chelants, proteins, skin active agents, sunscreens, UV absorbers, vitamins, niacinamide, caffeine, minoxidil, and combinations thereof. In at least one embodiment, the formulation comprises from 0 wt.-% to 5 wt.-% vitamins and amino acids, by total weight of the formulation. The formulation may also comprise pigment materials such as inorganic, nitroso, monoazo, diazo, carotenoid, triphenyl methane, triaryl methane, xanthene, quinoline, oxazine, azine, anthraquinone, indigoid, thionindigoid, quinacridone, phthalocyanine, botanical, natural colors, including: water soluble components such as those having C.I. Names. The formulation may comprise from 0 wt.-%, preferably from 0.0001 wt.-% to 5 wt.-% pigment materials.

In at least one embodiment, the formulation comprises an oily substance, which is any fatty substance which is liquid at room temperature (25° C.). In at least one embodiment, the formulation comprises oily substance selected from the group consisting of silicone oils, volatile or nonvolatile, linear, branched or cyclic, optionally with organic modification; phenylsilicones; silicone resins and silicone gums; mineral oils such as paraffin oil or vaseline oil; oils of animal origin such as perhydrosqualene, lanolin; oils of plant origin such as liquid triglycerides, e.g., sunflower oil, corn oil, soybean oil, rice oil, jojoba oil, babussu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, argan oil, abyssinian oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C₆-C₁₃) fatty acids with linear (C₆-C₂₀) fatty alcohols; esters of branched (C₆-C₁₃) carboxylic acids with linear (C₆-C₂₀) fatty alcohols, esters of linear (C₆-C₁₈) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C₆-C₁₀) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example;

fluorinated and perfluorinated oils; fluorinated silicone oils; mixtures of the aforementioned compounds.

In at least one embodiment, the formulation comprises a non-ionic coemulsifier. In at least one embodiment, the non-ionic coemulsifier is selected from adducts of from 0 to 30 mol of ethylene oxide and/or from 0 to 5 mol of propylene oxide with linear fatty alcohols having 8 to 22 carbon atoms, with fatty acids having 12 to 22 carbon atoms, with alkylphenols having 8 to 15 carbon atoms in the alkyl group, and with sorbitan or sorbitol esters; (C₁₂-C₁₈) fatty acid monoesters and diesters of adducts of from 0 to 30 mol of ethylene oxide with glycerol; glycerol monoesters and diesters and sorbitan monoesters and diesters of saturated and unsaturated fatty acids having 6 to 22 carbon atoms and, where appropriate, their ethylene oxide adducts; adducts of from 15 to 60 mol of ethylene oxide with castor oil and/or hydrogenated castor oil; polyol esters and especially polyglycerol esters, such as polyglyceryl polyricinoleate and polyglyceryl poly-12-hydroxystearate, for example. Likewise suitable are mixtures of compounds from one or more of these classes of substance. Examples of suitable ionogenic coemulsifiers include anionic emulsifiers, such as mono-, di- or tri-phosphoric esters, but also cationic emulsifiers such as mono-, di-, or tri-alkyl quats and their polymeric derivatives.

In at least one embodiment, the formulation comprises a cationic polymer. Suitable cationic polymers include those known under the INCI designation “Polyquaternium”, especially Polyquaternium-31, Polyquaternium-16, Polyquaternium-24. Polyquaternium-7, Polyquaternium-22, Polyquaternium-39. Polyquaternium-28. Polyquaternium-2, Polyquaternium-10, Polyquaternium-11, and also Polyquaternium 37 & mineral oil & PPG trideceth (Salcare SC95), PVP-dimethylaminoethyl methacrylate copolymer, guar-hydroxypropyltriammonium chlorides, and also calcium alginate and ammonium alginate. It is additionally possible to employ cationic cellulose derivatives; cationic starch; copolymers of diallylammonium salts and acrylamides; quaternized vinylpyrrolidone/vinylimidazole polymers; condensation products of polyglycols and amines: quaternized collagen polypeptides; quaternized wheat polypeptides; polyethyleneimines; cationic silicone polymers, such as amidomethicones, for example; copolymers of adipic acid and dimethylaminohydroxypropyldiethylenetriamine; polyaminopolyamide and cationic chitin derivatives, such as chitosan, for example.

In at least one embodiment, the formulation comprises a superfatting agent. As superfatting agents it is possible to use substances such as, for example, polyethoxylated lanolin derivatives, lecithin derivatives, polyol fatty acid esters, monoglycerides, or fatty acid alkanol amides, the latter serving simultaneously as foam stabilizers. Moisturizers available include for example isopropyl palmitate, glycerol and/or sorbitol.

In at least one embodiment, the formulation comprises a stabilizer. As stabilizer it is possible to use metal salts of fatty acids, such as magnesium, aluminum and/or zinc stearate, for example.

In at least one embodiment, the formulation comprises a care additive. The formulations can be blended with conventional ceramides, pseudoceramides, fatty acid N-alkylpolyhydroxyalkyl amides, cholesterol, cholesterol fatty acid esters, fatty acids, triglycerides, cerebrosides, phospholipids, panthenol and similar substances as a care additive.

In at least one embodiment, the formulation comprises an anti-fungal substance. In at least one embodiment, the anti-fungal substance is selected from the group consisting of ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, zinc pyrithione, piroctone olamine (octopirox), and combinations thereof. In at least one embodiment, the formulation comprises a total amount of anti-fungal substance in the formulation of from 0.1 wt.-% to 1 wt.-%. In at least one embodiment, the formulation comprises pyridinethione anti-dandruff particulates. For example, 1-hydroxy-2-pyridinethione salts are highly preferred particulate anti-dandruff agents. The concentration of pyridinethione antidandruff particulate may range from 0.1 wt.-% to 4 wt.-%, by total weight of the formulation, preferably from 0.1 wt.-% to 3 wt.-%, more preferably from 0.3 wt.-% to 2 wt.-%. Preferred pyridinethione salts include those formed from heavy metals such as zinc, tin, cadmium, magnesium, aluminum or zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form. Salts formed from other cations, such as sodium, may also be suitable.

Functional acids are acidic substances used to impart a clinical functionality to the skin or hair upon application. Suitable functional acids include alpha-hydroxy acids, beta-hydroxy acids, lactic acid, retinoic acid, and similar substances.

In at least one embodiment, the formulation comprises an astringent. In at least one embodiment, the astringent is selected from the group consisting of magnesium oxide, aluminum oxide, titanium dioxide, zirconium dioxide, zinc oxide, oxide hydrates, aluminum oxide hydrate (boehmite) and hydroxide, chlorohydrates of calcium, magnesium, aluminum, titanium, zirconium or zinc. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% astringent.

In at least one embodiment, the formulation comprises a deodorizing agent. In at least one embodiment, the deodorizing agent is selected from the group consisting of allantoin, bisabolol, and combinations thereof. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% deodorizing agent.

In at least one embodiment, the formulation comprises a sun protection agent and/or UV filter. Suitable sun protection agents and UV filters are disclosed in WO-2013/017262A1, from page 32, line 11 to the end of page 33. Suitable sun protection agents and UV filters are also disclosed in WO2018/002100 (pages 37-40).

In at least one embodiment, the formulation comprises an anti-oxidant. In at least one embodiment, the anti-oxidant is selected from the group consisting of amino acids, peptides, sugars, imidazoles, carotinoids, carotenes, chlorogenic acid, lipoic acid, thiols, thiol glycosyl esters, thiol N-acetyl esters, thiol methyl esters, thiol ethyl esters, thiol propyl esters, thiol amyl esters, thiol butyl esters, thiol lauryl esters, thiol palmitoyl esters, thiol oleyl esters, thiol linoleyl esters, thiol cholesteryl esters, thiol glyceryl esters, dilaurylthiodipropionate, distearylthiodipropionate, thiodipropionic acid, metal chelators, hydroxy acids, fatty acids, folic acids, vitamin C, tocopherol, vitamin A, stilbenes, derivatives and combinations thereof. In at least one embodiment, the anti-oxidant is selected from the group consisting of glycine, histidine, tyrosine, tryptophan, urocaninic acid, D,L-carnosine. D-carnosine, L-carnosine, beta-carotene, alpha-carotene, lycopene, dihydrolipoic acid, aurothioglucose, propylthiouracil, thioredoxine, glutathione, cysteine, cystine, cystamine, buthioninsulfoximine, homocysteinsulfoximine, buthioninsulfone, penta-, hexa-, heptathioninsulfoximine, hydroxyfatty acids, palmitic acid, phytinic acid, lactoferrin, citric acid, lactic acid, malic acid, humic acid, bile acid, bilirubin, biliverdin, EDTA. EGTA, linoleic acid, linolenic acid, oleic acid, butylhydroxyanisol, trihydroxybutyrophenone, ubichinon, ubichinol, ascorbylpalmitate. Mg-ascorbylphosphate, ascorbylacetate, vitamin E acetate, vitamin A palmitate, carnosine, mannose, ZnO, ZnSO4, selenium methionine, stilbenes, superoxide dismutase, and combinations thereof. In at least one embodiment, the antioxidant is selected from the group consisting of vitamin A, vitamin A derivatives, vitamin E, vitamin E derivatives, and combinations thereof. In at least one embodiment, the formulation comprises from 0.001 wt.-% to 10 wt.-%, preferably from 0.05 wt.-% to 5 wt.-%, particularly preferably from 0.1 wt.-% to 3 wt.-%, also particularly preferably from 0.05 wt.-% to 1 wt.-% antioxidant.

In at least one embodiment, the formulation comprises a dye or pigment. In at least one embodiment, the formulation comprises at least one pigment. Suitable dyes and pigments are disclosed in WO2013/017262A1 in the table spanning pages 36 to 43. These may be colored pigments which impart color effects to the product mass or to hair, or they may be luster effect pigments which impart luster effects to the product mass or to hair. The color or luster effects on hair are preferably temporary, i.e. they last until the next hair wash and can be removed again by washing the hair with customary shampoos. In at least one embodiment, the formulation comprises a total amount of from 0.01 wt.-% to 25 wt.-%, preferably from 5 wt.-% to 15 wt.-% pigment. In at least one embodiment, the particle size of the pigment is from 1 micron to 200 micron, preferably from 3 micron to 150 micron, more preferably from 10 micron to 100 micron. The pigments are colorants which are virtually insoluble in the application medium, and may be inorganic or organic. Inorganic-organic mixed pigments are also possible. Preference is given to inorganic pigments. The advantage of inorganic pigments is their excellent resistance to light, weather and temperature. The inorganic pigments may be of natural origin. In at least one embodiment, the inorganic pigment is selected from the group consisting of chalk, ochre, umber, green earth, burnt sienna, graphite, and combinations thereof. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, where preferably at least one pigment is a colored, nonwhite pigment. In at least one embodiment, the pigment is selected from the group consisting of metal oxides, hydroxides and oxide hydrates, mixed phase pigments, sulfur-containing silicates, metal sulfides, complex metal cyanides, metal sulfates, chromates and molybdates, and the metals themselves (bronze pigments), and combinations thereof. In at least one embodiment, the pigment is selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), and combinations thereof. In at least one embodiment, the pigment is selected from the group consisting of pearlescent and colored pigments based on mica which are coated with a metal oxide or a metal oxychloride, such as titanium dioxide or bismuth oxychloride, and optionally further color-imparting substances, such as iron oxides, Prussian blue, ultramarine, carmine etc. and where the color can be determined by varying the layer thickness. Such pigments are sold, for example, under the trade names Rona®, Colorona®, Dichrona® and Timiron® by Merck. In at least one embodiment, the pigment is selected from the group consisting of organic pigments such as sepia, gamboge, bone charcoal, Cassel brown, indigo, chlorophyll and other plant pigments. In at least one embodiment, the pigment is selected from the group consisting of synthetic organic pigments such as azo pigments, anthraquinoids, indigoids, dioxazine, quinacridone, phthalocyanine, isoindolinone, perylene and perinone, metal complex, alkali blue and diketopyrrolopyrrole pigments.

In at least one embodiment, the formulation comprises from 0.01 wt.-% to wt.-%, preferably from 0.05 wt.-% to 5 wt.-%, of at least one particulate substance. Suitable substances are, for example, substances which are solid at room temperature (25° C.) and are in the form of particles. In at least one embodiment, the particulate substance is selected from the group consisting of silica, silicates, aluminates, clay earths, mica, insoluble salts, in particular insoluble inorganic metal salts, metal oxides, e.g. titanium dioxide, minerals and insoluble polymer particles are suitable. The particles may be present in the formulation in undissolved, preferably stably dispersed form, and, following application to the keratin substrate and evaporation of the solvent, can deposit on the substrate in solid form. A stable dispersion can be achieved by providing the formulation with a yield point which is large enough to prevent the solid particles from sinking. An adequate yield point can be established using suitable gel formers in a suitable amount. In at least one embodiment, the particulate substance is selected from the group consisting of silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

In at least one embodiment, the formulation comprises a direct dye. Preferred among the direct dyes are the following compounds, alone or in combination with one another: Hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydroxyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4 {[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof.

Particularly preferred among the aforesaid direct dyes are the following compounds, alone or in combination with one another: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydro-xyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)-amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof. In at least one embodiment, the total quantity of direct dyes in the formulation amounts to 0.01 to 15 wt.-%, preferably 0.1 to wt.-%, most preferred 0.5 to 8 wt.-%.

In at least one embodiment, the formulation comprises a conditioning agent. In at least one embodiment, the conditioning agent is a water insoluble, water dispersible, non-volatile, liquid that forms emulsified, liquid particles. In at least one embodiment, the conditioning agent is a silicone (e.g., silicone oil, cationic silicone, silicone gum, high refractive silicone, or silicone resin), an organic conditioning oil (e.g., hydrocarbon oils, polyolefins, or fatty esters), a cationic conditioning surfactant, a high melting point fatty compound, or combinations thereof.

In at least one embodiment, the conditioning agent is a silicone, and the formulation comprises from 0.01% to 10%, or from 0.1% to 5% silicone conditioning agent, by total weight of the formulation. Suitable silicone conditioning agents, and optional suspending agents for the silicone, are described in U.S. Pat. No. 5,104,646. In at least one embodiment, the formulation comprises a silicone gum selected from the group consisting of polydimethylsiloxane, (polydimethylsiloxane) (methylvinylsiloxane) copolymer, poly(dimethylsiloxane) (diphenylsiloxane) (methylvinylsiloxane) copolymer, and mixtures thereof.

In at least one embodiment, the formulation comprises a terminal aminosilicone. “Terminal aminosilicone” as defined herein means silicone comprising one or more amino groups at one or both ends of the silicone backbone. In at least one embodiment, the formulation is substantially free of any silicone compound comprising pendant amino groups. In an embodiment, the formulation is substantially free of any silicone compound other than terminal aminosilicones. In at least one embodiment, the amino group at at least one terminus of the silicone backbone of the terminal aminosilicone is selected from the group consisting of primary amines, secondary amines and tertiary amines. In at least one embodiment, the formulation comprises a terminal aminosilicone conforming to Formula (S):

(R^{F})ₐG₃₋ₐ-Si—(—OSiG₂)ₙ-O-SiG₃₋ₐ(R^{F})ₐ (S)
wherein
G is hydrogen, phenyl, hydroxy, or C₁-C₈ alkyl, preferably methyl;
a is an integer having a value from 1 to 3, preferably 1;
b is 0, 1 or 2, preferably 1;
n is a number from 0 to 1,999;
R^{F} is a monovalent radical conforming to the general formula C_{q}H_{2q}L,
   wherein q is an integer having a value from 2 to 8 and L is selected from the following groups: —N(R^{T})CH₂—CH₂—N(R^{T})₂; —N(R^{T})₂; —N(R^{T})₃A⁻;
   —N(R^{T})CH₂—CH₂—NR^{T}H₂A⁻; wherein R^{T} is hydrogen, phenyl, benzyl, or a saturated hydrocarbon radical, preferably an alkyl radical having from 1 to 20 carbon atoms; A⁻ is a halide ion.

In at least one embodiment, the terminal aminosilicone corresponding to Formula (S) has a=1, q=3, G=methyl, n is from 1000 to 2500, alternatively from 1500 to 1700, and L is-N(CH₃)₂. A suitable terminal aminosilicone corresponding to Formula (S) has a=O. G=methyl, n is from 100 to 1500, or from 200 to 800, and L is selected from the following groups: —N(R^{T})CH₂—CH₂—N(R^{T})₂; —N(R^{T})₂; —N(R^{T})₃A⁻; —N(R^{T})CH₂—CH₂—NR^{T}H₂A⁻; wherein R^{T} is hydrogen, phenyl, benzyl, or a saturated hydrocarbon radical, preferably an alkyl radical from having from 1 to 20 carbon atoms; A: is a halide ion, alternatively L is —NH₂. In at least one embodiment, the terminal aminosilicone is selected from the group consisting of bis-aminomethyl dimethicone, bis-aminoethyl dimethicone, bis-aminopropyl dimethicone, bis-aminobutyl dimethicone, and mixtures thereof. In an embodiment, the viscosity of the terminal aminosilicone is from 1,000 to 30,000 cPs, or from 5,000 to 20,000 cPs, measured at 25° C. In at least one embodiment, the formulation comprises from 0.1% to 20%, or from 0.5% to 10%, or from 1% to 6% terminal aminosilicone, by total weight of the formulation.

In at least one embodiment, the formulation comprises a high melting point fatty compound. The high melting point fatty compound has a melting point of 25° C. or higher. In at least one embodiment, the high melting point fatty compound is selected from the group consisting of a fatty alcohol, fatty acid, fatty alcohol derivative, fatty acid derivative, and mixtures thereof. Non-limiting examples of the high melting point compounds are found in International Cosmetic Ingredient Dictionary, Fifth Edition, 1993, and CTFA Cosmetic Ingredient Handbook, Second Edition, 1992. The formulation may comprise from 0.1% to 40%, or from 1% to 30%, or from 1.5% to 16%, or from 1.5% to 8% of a high melting point fatty compound, by total weight of the formulation. This is advantageous in view of providing improved conditioning benefits such as slippery feel during the application to wet hair, softness and moisturized feel on dry hair. In at least one embodiment, the fatty alcohol is selected from the group consisting of: cetyl alcohol, stearyl alcohol, behenyl alcohol, and mixtures thereof. In at least one embodiment, the formulation comprises a linear fatty alcohol, wherein the linear fatty alcohol is comprised in a lamellar gel matrix. A lamellar gel matrix is suitable for providing various conditioning benefits such as slippery feel during the application to wet hair and softness and moisturized feel on dry hair. The linear fatty alcohol may comprise from 8 to 24 carbon atoms. In an embodiment, the linear fatty alcohol is selected from the group consisting of: cetyl alcohol, stearyl alcohol, and mixtures thereof. In an embodiment, the weight ratio of total linear fatty alcohol to terminal aminosilicone is from 0.5:1 to 10:1, or from 1:1 to 5:1, or from 2.4:1 to 2.7:1.

In at least one embodiment, the lamellar gel matrix comprises a cationic conditioning surfactant and a high melting point fatty compound. In view of providing the lamellar gel matrix, the cationic conditioning surfactant and the high melting point fatty compound are contained at a level such that the weight ratio of the cationic surfactant to the high melting point fatty compound is in the range of from 1:1 to 1:10, or from 1:1 to 1:6.

In at least one embodiment, the formulation comprises a cationic conditioning surfactant. In at least one embodiment, the formulation comprises from 0.05% to 3.0%, or from 0.075% to 2.0%, or from 0.1% to 1.0%, of cationic conditioning surfactant by total weight of the formulation. In at least one embodiment, the cationic conditioning surfactant is comprised in a lamellar gel matrix. In other words, the formulation comprises a lamellar gel matrix and the lamellar gel matrix comprises the cationic conditioning surfactant. In an embodiment, cationic conditioning surfactant is according to Formula (C):

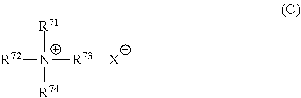

wherein at least one of R⁷¹, R⁷², R⁷³ and R⁷⁴ is selected from an aliphatic group of from 8 to 30 carbon atoms, an aromatic, alkoxy, polyoxyalkylene, alkylamido, hydroxyalkyl, aryl, or an alkylaryl group having up to 22 carbon atoms; the remainder of R⁷¹, R⁷², R⁷³ and R⁷⁴ are independently selected from the group consisting of an aliphatic group of from 1 to 22 carbon atoms, and an aromatic, alkoxy, polyoxyalkylene, alkylamido, hydroxyalkyl, aryl or alkylaryl group having up to 22 carbon atoms;
X is selected from the group consisting of: halogen, acetate, citrate, lactate, glycolate, phosphate, nitrate, sulfonate, sulfate, alkylsulfate, alkyl sulfonate, and combinations thereof.

In at least one embodiment, the cationic conditioning surfactant is selected from the group consisting of behenyl trimethyl ammonium chloride, methyl sulfate or ethyl sulfate, and stearyl trimethyl ammonium chloride, methyl sulfate or ethyl sulfate. It is believed that a longer alkyl group provides improved smoothness and soft feeling on wet and dry hair, compared to cationic surfactants with a shorter alkyl group. It is also believed that such cationic surfactants can provide reduced irritation, compared to those having a shorter alkyl group.

In at least one embodiment, the cationic surfactant is a di-long alkyl quaternized ammonium salt selected from the group consisting of: dialkyl (C14-C18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, and mixtures thereof.

In at least one embodiment, the cationic surfactant is a tertiary amido amine having an alkyl group of from 12 to 22 carbons. The tertiary amido amine may be selected from the group consisting of stearamidopropyldimethyl-, stearamidopropyldiethyl-, stearamidoethyldiethyl-, stearamidoethyldimethyl-, palmitamidopropyldimethyl-, palmitamidopropyldiethyl-, palmitamidoethyldiethyl-, palmitamidoethyldimethyl-, behenamidopropyldimethyl-, behenamidopropyldiethyl-, behenamidoethyldiethyl-, behenamidoethyldimethyl-, arachidamidopropyldimethyl-, arachidamidopropyldiethyl-, arachidamidoethyldiethyl-, and arachidamidoethyldimethyl-amine, diethylaminoethylstearamide, and mixtures thereof. A tertiary amido amine may be used in combination with an acid. The acid is typically used as a salt-forming anion. In an embodiment, the acid is selected from the group consisting of lactic acid, malic acid, hydrochloric acid, 1-glumatic acid, acetic acid, citric acid, and mixtures thereof.

In at least one embodiment, the cationic surfactant is selected from the group consisting of cetyltrimonium chloride (CTAC), stearyltrimonium chloride (STAC), behentrimonium methosulfate, stearoylamidopropyldimethyl amine (SAPDMA), distearyldimethylammonium chloride, and mixtures thereof.

In at least one embodiment, the formulation comprises a surfactant system. In at least one embodiment, the surfactant system comprises a surfactant selected from the group consisting of anionic surfactants, cationic surfactants, non-ionic surfactants, zwitterionic surfactants and/or amphoteric surfactants. In at least one embodiment, the formulation comprises a total amount of surfactant of from 0.01 wt.-% to 70 wt.-%, from 0.1 wt.-% to 40%, from 1 wt.-% to 30%, from 2 wt.-% to 20 wt.-%.

In at least one embodiment, the formulation comprises an anionic surfactant. In at least one embodiment, the anionic surfactant is selected from the group consisting of (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, alpha-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono, di-, and triethanolammonium, and analogous alkylammonium salts. In at least one embodiment, the anionic surfactant is the salt of an anionic surfactant comprising 12 to 14 carbon atoms. In at least one embodiment, the anionic surfactant is selected from the group consisting of sodium lauryl sulfate, sodium laureth sulfate, sodium tridecyl sulfate, sodium trideceth sulfate, sodium myristyl sulfate, sodium myreth sulfate, and mixtures thereof.

In at least one embodiment, the formulation comprises an acylglycinate surfactant.

In at least one embodiment, the acylglycinate surfactant conforms to the formula (Y):

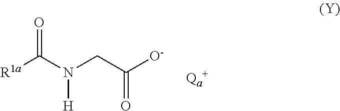

wherein
R^{1a} is a linear or branched, saturated alkyl group having 6 to 30, preferably 8 to 22, particularly preferably 8 to 18 carbon atoms or is a linear or branched, mono- or polyunsaturated alkenyl group having 6 to 30, preferably 8 to 22, particularly preferably 12 to 18 carbon atoms, and
Qₐ⁺ is a cation.

In at least one embodiment, Qₐ⁺ is selected from the group consisting of Li⁺, Na⁺, K⁺, Mg⁺⁺, Ca⁺⁺, Al⁺⁺⁺, NH₄⁺, a monoalkylammmonium ion, a dialkylammonium ion, a trialkylammonium ion and a tetraalkylammonium ion, or combinations thereof. In at least one embodiment, the acylglycinate surfactant is selected from sodium cocoylglycinate and potassium cocoylglycinate. In at least one embodiment, the acylglycinate surfactant is selected from those conforming to formula (Y), wherein R is C₁₂ alkyl or C₁₄ alkyl. In at least one embodiment, the acylglycinate surfactant is selected from those conforming to formula (Y), wherein R is C₁₆ alkyl or C₁₈ alkyl.

In at least one embodiment, the formulation comprises a glutamate surfactant corresponding to formula (Z) or a salt thereof:

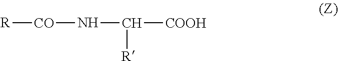

wherein
R′ is HOOC—CH2-CH2- or M⁺-OOC—CH2-CH2- wherein M⁺ is a cation;
R is a linear or branched, saturated alkyl group having 6 to 30, preferably 8 to 22, more preferably 8 to 18 carbon atoms or is a linear or branched, mono- or polyunsaturated alkenyl group having 6 to 30, preferably 8 to 22, more preferably 12 to 18 carbon atoms. In at least one embodiment, M⁺ is a metal cation. In at least one embodiment, M⁺ is selected from the group consisting of Li⁺, Na⁺, K⁺, Mg⁺⁺, Ca⁺⁺, Al⁺⁺⁺, NH₄⁺, a monoalkylammmonium ion, a dialkylammonium ion, a trialkylammonium ion and a tetraalkylammonium ion, or combinations thereof.

In at least one embodiment, the glutamate surfactant is selected from sodium cocoyl glutamate and potassium cocoyl glutamate. In at least one embodiment, the glutamate surfactant is selected from those conforming to formula (Z), wherein R is C₁₂ alkyl or C₁₄ alkyl. In at least one embodiment, the glutamate surfactant is selected from those conforming to formula (Z), wherein R is C₁₆ alkyl or C₁₈ alkyl.

In at least one embodiment, the formulation comprises from 0.01 wt.-% to 30 wt.-%, preferably from 1 wt.-% to 25 wt.-%, more preferably from 5 wt.-% to 20 wt.-%, particularly preferably from 12 wt.-% to 18 wt.-% anionic surfactant.

In at least one embodiment, the formulation comprises a non-ionic surfactant. In at least one embodiment, the non-ionic surfactant has an HLB (Hydrophilic Lipophilic Balance) of greater than 12. Optionally, the non-ionic surfactant is selected from the group consisting of ethoxylated or ethoxylated/propoxylated fatty alcohols with a fatty chain having 12 to 22 carbon atoms, ethoxylated sterols, such as stearyl- or lauryl alcohol (EO-7), PEG-16 soya sterol or PEG-10 soya sterol, polyoxyethylene polyoxypropylene block polymers (poloxamers), and mixtures thereof.

In at least one embodiment, the non-ionic surfactant is selected from the group consisting of ethoxylated fatty alcohols, fatty acids, fatty acid glycerides or alkylphenols, in particular addition products of from 2 to 30 mol of ethylene oxide and/or 1 to 5 mol of propylene oxide onto C₈- to C₂₂-fatty alcohols, onto C₁₂- to C₂₂-fatty acids or onto alkyl phenols having 8 to 15 carbon atoms in the alkyl group, C₁₂- to C₂₂-fatty acid mono- and diesters of addition products of from 1 to 30 mol of ethylene oxide onto glycerol, addition products of from 5 to 60 mol of ethylene oxide onto castor oil or onto hydrogenated castor oil, fatty acid sugar esters, in particular esters of sucrose and one or two C₈- to C₂₂-fatty acids, INCI: Sucrose Cocoate. Sucrose Dilaurate, Sucrose Distearate, Sucrose Laurate, Sucrose Myristate, Sucrose Oleate, Sucrose Palmitate, Sucrose Ricinoleate, Sucrose Stearate, esters of sorbitan and one, two or three C₈- to C₂₂-fatty acids and a degree of ethoxylation of from 4 to 20, polyglyceryl fatty acid esters, in particular of one, two or more C₈- to C₂₂-fatty acids and polyglycerol having preferably 2 to 20 glyceryl units, alkyl glucosides, alkyl oligoglucosides and alkyl polyglucosides having C₈ to C₂₂-alkyl groups, e.g. decylglucoside or laurylglucoside, and mixtures thereof.

In at least one embodiment, the non-ionic surfactant is selected from the group consisting of fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates (e.g. Pluronics®), fatty acid alkylol amides (fatty acid amide polyethylene glycols), N-alkyl-, N-alkoxypolyhydroxy-fatty acid amides, sucrose esters, sorbitol esters, polyglycol ethers, and mixtures thereof.

In at least one embodiment, the formulation comprises a fatty N-methyl-N-glucamide surfactant, wherein the fatty N-methyl-N-glucamide surfactant conforms to the formula (X):

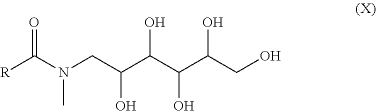

wherein R is selected from saturated or unsaturated hydrocarbon chains having 5 to 23 carbon atoms. Preferably, R in formula (X) is selected from saturated or unsaturated hydrocarbon chains having 7 to 17 carbon atoms. Also preferably, the R—C═O residue in formula (X) is derived from caprylic acid, capric acid, lauric acid, myristic acid, palmitic acid, stearic acid, oleic acid, linoleic acid, linolenic acid, coconut fatty acids, or mixtures thereof. Also preferably, the R—C═O residue in formula (X) is derived from 9-decenoic acid, 9-dodecenoic acid, or mixtures thereof.

Particularly preferred N-methyl-N-acylglucamines of formula (X) are capryloyl/caproyl methyl glucamide, lauroyl/myristoyl methyl glucamide, cocoyl methyl glucamide, oleyl methyl glucamide, or mixtures thereof. Such N-methyl-N-acylglucamines are commercially available from Clariant (GlucoTain® Clear, GlucoTain® Plus, GlucoTain® Flex, GlucoTain® Care, GlucoTain® Sense).

Also particularly preferred N-methyl-N-acylglucamines of formula (X) are N-9-decenoyl-N-methylglucamine, N-9-dodecenoyl-N-methylglucamine, or mixtures thereof.

In at least one embodiment, the formulation comprises from 1 wt.-% to 20 wt.-%, preferably from 2 wt.-% to 10 wt.-%, more preferably from 3 wt.-% to 7 wt.-% non-ionic surfactant.

In at least one embodiment, the amphoteric surfactants are selected from the group consisting of N—(C₁₂-C₁₈)-alkyl-β-aminopropionates and N—(C₁₂-C₁₈)-alkyl-β-iminodipropionates as alkali metal salts and mono-, di-, and trialkylammonium salts; N-acylaminoalkyl-N,N-dimethylacetobetaine, preferably N—(C₈-C₁₈)-acylaminopropyl-N, N-dimethylacetobetaine, (C₁₂-C₁₈)-alkyl-dimethyl-sulfopropylbetaine, amphosurfactants based on imidazoline (e.g. Miranol® Steinapon®), preferably the sodium salt of 1-(beta-carboxymethyloxyethyl)-1-(carboxymethyl)-2-laurylimidazolinium; amine oxides, e.g. (C₁₂-C₁₈)-alkyldimethylamine oxides, fatty acid amidoalkyldimethylamine oxides, and mixtures thereof.

In at least one embodiment, the formulation comprises a betaine surfactant. Optionally, the betaine surfactant is selected from C₈- to C₁₈-alkylbetaines. In at least one embodiment, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine, and combinations thereof. Optionally, the betaine surfactant is selected from C₈- to C₁₈-sulfobetaines. In at least one embodiment, the betaine surfactant is selected from the group consisting of cocodimethylsulfopropylbetaine, stearyldimethylsulfopropylbetaine, lauryldimethylsulfoethylbetaine, laurylbis(2-hydroxyethyl) sulfopropylbetaine, and combinations thereof. Optionally, the betaine surfactant is selected from carboxyl derivatives of imidazole, the C₈- to C₁₈-alkyldimethylammonium acetates, the C₈- to C₁₈-alkyldimethylcarbonylmethylammonium salts, and the C₈- to C₁₈-fatty acid alkylamidobetaines, and mixtures thereof. Optionally, the C₈- to C₁₈-fatty acid alkylamidobetaine is selected from coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof.

In at least one embodiment, the formulation comprises from 0.5 wt.-% to 20 wt.-%, preferably from 1 wt.-% to 10 wt.-% amphoteric surfactant.

In at least one embodiment, the formulation comprises a surfactant system. In at least one embodiment, the surfactant system comprises at least one surfactant selected from the group consisting of lauryl sulfate, laureth sulfate, cocoamido-propylbetaine, sodium cocoylglutamate, lauroamphoacetate, and mixtures thereof. In at least one embodiment, the surfactant system comprises sodium laureth sulphate, sodium lauryl sulphate, and optionally cocamidopropyl betaine. In at least one embodiment, the surfactant system comprises sodium laureth sulphate, potassium cocoyl glutamate, and cocamidopropyl betaine.

In at least one embodiment, the formulation further comprises a hairstyling polymer. In at least one embodiment, the hairstyling polymer is selected from the group consisting of: amphoteric hairstyling polymers, zwitterionic hairstyling polymers, anionic hairstyling polymers, non-ionic hairstyling polymers, cationic hairstyling polymers, and mixtures thereof. In at least one embodiment, the formulation comprises from 0.01% to 20%, or from 0.01% to 16%, or from 0.01% to 10%, or from 1% to 8%, or from 2% to 6% of hairstyling polymer. Suitable hairstyling polymers are disclosed in, e.g., WO2018/002100 (pages 55-63).

In at least one embodiment, the formulation has a viscosity of from 0 cPs to 20,000 cPs. In at least one embodiment, the formulation has a viscosity of from 0.1 cPs to 10,000 cPs, or from 1 cPs to 5,000 cPs, or from 5 cPs to 3,500 cPs.

The viscosity measurement conditions are defined in the definitions section above. Viscosity may be important for anti-drip reasons. Dripping can be inconvenient for the user. Furthermore, more viscous formulations can be useful for measured dispensing. In at least one embodiment, the formulation has a viscosity of from 0 cPs to 1,000 cPs. This viscosity range is advantageous when the formulation is in the form of a facial cleanser in view of the need for distribution on skin and ability to rinse off.

In at least one embodiment, the formulation further comprises a viscosity-modifying substance. The viscosity-modifying substance is preferably a thickening polymer.

In at least one embodiment, the thickening polymer is a polymer based on acrylamidomethylpropanesulfonic acid (AMPS®). These polymers, even at pH values of 7 or less, exhibit good thickening performance. Especially preferably, the thickening polymer is selected from the group consisting of homo- or copolymers of acrylamidomethylpropanesulfonic acid and salts thereof. Among the polymers just mentioned, preference is given to polymers having at least 20 mol-% of units based on acrylamidomethylpropanesulfonic acid and/or salts thereof, and particular preference to polymers having at least 50 mol-% of units based on acrylamidomethylpropanesulfonic acid and/or salts thereof, the mole figures relating in each case to the overall polymer. In the case of the copolymers, in addition to structural units based on acrylamidomethylpropanesulfonic acid and/or salts thereof, preferably one or more structural units based on the following comonomers are present in the copolymers: acrylic acid, methacrylic acid, acrylamide, dimethylacrylamide, vinylpyrrolidone (VP), hydroxyethyl acrylate, hydroxyethyl methacrylate, acrylic or methacrylic esters of ethoxylated alcohols RO—(CH₂CH₂O)ₘH, in which R is an alkyl radical having 12 to 30 carbon atoms and m is a number from 3 to 30, and CH₂═CH—COO—(CH₂CH₂—COO)ₙX, in which n is a number from 0 to 10 and X is a counterion and is preferably H⁺, Na⁺ and/or NH₄⁺. The polymers selected from the group consisting of homo- or copolymers of acrylamidomethylpropanesulfonic acid and salts thereof may be crosslinked or non-crosslinked. In the case of crosslinking, they contain structural units based on monomers having 2 or more olefinic double bonds. In the case of crosslinking, preferably from 0.1 to 10 mol-% of such structural units are present in the homo- or copolymers, based on the overall polymer. If one or more structural units based on acrylamidomethylpropanesulfonic acid and/or salts thereof in the homo- or copolymers of acrylamidomethylpropanesulfonic acid and/or salts thereof have one or more counterions other than H⁺, these other counterions are preferably selected from the group consisting of Na⁺ and NH₄⁺. Suitable polymers are mentioned in publications including EP-0816403, EP-1069142, EP-1116733 and DE-10 2009 014877 (Clariant), EP-1347736 (L'Oréal) or EP-1496081 (Seppic). Examples include: Aristoflex® AVC (Ammonium Acryloyldimethyltaurate/VP Copolymer), Aristoflex® AVS (Sodium Acryloyldimethyltaurate/VP Crosspolymer), Aristoflex® TAC (Ammonium Acryloyl Dimethyltaurate Carboxyethyl Acrylate Crosspolymer), Hostacerin® AMPS (Ammonium Polyacryloyldimethyl Taurate), Aristoflex® HMB (Ammonium Acryloyldimethyltaurate/Beheneth-25 Methacrylate Crosspolymer), Aristoflex® BLV (Ammonium Acryloyldimethyltaurate/Beheneth-25 Methacrylate Crosspolymer), Aristoflex® HMS (Ammonium Acryloyldimethyltaurate/Steareth-Methacrylate Crosspolymer), Aristoflex® SNC (Ammonium Acryloyldimethyltaurate/Steareth-8 Methacrylate Copolymer), Aristoflex® LNC (Ammonium Acryloyldimethyltaurate/Laureth-7 Methacrylate Copolymer) or Sepinov® EMT 10 (Hydroxyethyl Acrylate/Sodium Acryloyldimethyl Taurate Copolymer). Sepigel® 305.

In at least one embodiment, the thickening polymer is selected from the group consisting of: copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C₁₀- to C₃₀-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C₁₀- to C₃₀-alcohols, and a third monomer type, which is chosen from C₁- to C₄-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C₂-, C₃- or C₄-alkylene and styrene; polyurethanes: hydroxypropyl starch phosphate; polyacrylamide; copolymers of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer); and mixtures thereof.

In at least one embodiment, the formulation has a pH value of from 2.0 to 12.0, preferably from 3.0 to 9.0, more preferably from 4.5 to 7.5. By varying the pH value, a formulation can be made available that is suitable for different applications.

In at least one embodiment, the formulation comprises an alkalizing agent or pH adjusting agent. In at least one embodiment, ammonia or caustic soda is suitable, but water-soluble, physiologically tolerable salts of organic or inorganic bases can also be considered. Optionally, the pH adjusting agent is selected from ammonium hydrogen carbonate, ammonia, monoethanolamine, ammonium carbonate. In at least one embodiment, the alkalizing agent or pH adjusting agent is selected from the group consisting of 2-amino-2-methyl-1-propanol, 2-amino-2-methyl-1,3-propanediol, 2-amino-2-ethyl-1,3-propanediol, tris(hydroxymethyl)-aminomethane. 2-amino-1-butanol, tris(2-hydroxypropyl)-amine, 2,2-iminobisethanol, lysine, iminourea (guanidine carbonate), tetrahydro-1,4-oxazine, 2-amino-5-guanidin-valeric acid, 2-aminoethansulfonic acid, diethanolamine, triethanolamine, N-methyl ethanolamine, isopropanolamine, diisopropanolamine, triisopropanolamine, glucamine, sodium hydroxide, potassium hydroxide, lithium hydroxide, magnesium oxide, and mixtures thereof.

To establish an acidic pH value, an acid can be included. In at least one embodiment, the formulation comprises an acid selected from the group consisting of hydrochloric acid, phosphoric acid, acetic acid, formic acid, sulfuric acid, citric acid, and mixtures thereof. Citric acid is most preferred in that it has high consumer acceptance. In at least one embodiment, the acidic pH is adjusted with a buffer such as a phosphate buffer, a TRIS buffer or a citric buffer. The buffers may be used alone or in combination with an acid.

In at least one embodiment, the formulation is in liquid form. In an alternative embodiment, the formulation is in solid form. Optionally, the formulation is in powdered or granulated form. This is advantageous in that it is not needed to ship liquid, which is typically heavy, over long distances, which has economic and environmental benefits. A solid form can be achieved by spray drying the formulation or by using a rotary evaporator. A solid form can also be achieved by extrusion or pressing. The formulation can be converted into liquid form after it has been shipped, e.g. by adding water.

In at least one embodiment, the formulation is a household cleaning formulation. In a preferred embodiment, the household cleaning formulation is a hand dishwashing formulation or a hard surface cleaner.

In at least one embodiment, the formulation is a hand dishwashing formulation. In at least one embodiment, the hand dishwashing formulation comprises an anionic surfactant. In at least one embodiment, the hand dishwashing formulation comprises from 5 wt.-% to 25 wt.-% anionic surfactant. In at least one embodiment, the hand dishwashing formulation comprises a surfactant system comprising at least one anionic surfactant and a further surfactant selected from non-ionic surfactants, amphoteric surfactants, zwitterionic surfactants, and combinations thereof.

Preferably, the hand dishwashing formulation comprises cocoamidopropylbetaine or an amine oxide. Preferably, the amine oxide is lauryl amine oxide, cocoyl amine oxide, or a combination thereof. In at least one embodiment, the pH value of the hand dishwashing formulation is between pH 5.0 and pH 10, preferably between pH 5.5 and pH 9.0. In the case of the hand dishwashing formulation comprising an amine oxide, the hand dishwashing formulation preferably has a pH of between pH 7.5 and pH 9.5, most preferably between pH 8.0 and pH 9.0.

In at least one embodiment, the formulation is a hard surface cleaner. In at least one embodiment, the hard surface cleaner comprises an anionic surfactant. In at least one embodiment, the hard surface cleaner comprises from 1 wt.-% to 10 wt.-% anionic surfactant. In at least one embodiment, the hard surface cleaner comprises a nonionic surfactant. In at least one embodiment, the hard surface cleaner comprises from 1 wt.-% to 10 wt.-% nonionic surfactant. In at least one embodiment, the hard surface cleaner comprises a surfactant system comprising at least one anionic surfactant and a further surfactant selected from non-ionic surfactants, amphoteric surfactants, zwitterionic surfactants, and combinations thereof. Preferably, the hard surface cleaner comprises linear alkylbenzene sulfonate and fatty alcohol ethoxylate. In at least one embodiment, the pH value of the hard surface cleaner is between pH 5.0 and pH 11, preferably between pH 6.0 and pH 9.0.

In at least one embodiment, the formulation is a liquid laundry detergent formulation comprising one or more surfactants. Preferably, the one or more surfactants of the liquid laundry detergent formulation are selected from the group consisting of anionic, nonionic, cationic and zwitterionic surfactants, and more preferably from the group consisting of anionic, nonionic and zwitterionic surfactants.

### Anionic Surfactants

In at least one embodiment, the formulation comprises an anionic surfactant. Anionic surfactants are particularly useful in cleaning formulations such as household cleaning formulations. Preferred anionic surfactants are alkyl sulfonates and alkyl ether sulfates. Preferred alkyl sulfonates are alkylbenzene sulfonates, particularly linear alkylbenzene sulfonates (LAS) having an alkyl chain length of C₈-C₁₅, preferably C₁₂-C₁₄. Possible counterions for concentrated alkaline liquids are ammonium ions, e.g. those generated by the neutralization of alkylbenzene sulfonic acid with one or more ethanolamines, for example monoethanolamine (MEA) and triethanolamine (TEA), or alternatively, alkali metals, e.g. those arising from the neutralization of alkylbenzene sulfonic acid with alkali hydroxides. Preferred alkyl ether sulfates (AES) are alkyl polyethoxylate sulfate anionic surfactants.

### Nonionic Surfactants

In at least one embodiment, the formulation comprises a nonionic surfactant. Nonionic surfactants include primary and secondary alcohol ethoxylates, especially C₈-C₂₀ aliphatic alcohols ethoxylated with an average of from 1 to 20 moles of ethylene oxide per mole of alcohol, and more especially the C₁₀-C₁₅ primary and secondary aliphatic alcohols ethoxylated with an average of from 1 to 10 moles of ethylene oxide per mole of alcohol. Non-ethoxylated nonionic surfactants include alkyl polyglycosides, glycerol monoethers and polyhydroxy amides (glucamides). Mixtures of nonionic surfactants may also be used.

When included therein, the household cleaning formulation, particularly the liquid laundry detergent formulation, preferably comprises from 0.2 wt.-% to 40 wt.-%, more preferably from 1 wt.-% to 20 wt.-% nonionic surfactant, such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, N-acyl N-alkyl derivatives of glucosamine (glucamides), or combinations thereof. Nonionic surfactants that may be used include the primary and secondary alcohol ethoxylates, especially the C₈-C₂₀ aliphatic alcohols ethoxylated with an average of from 1 to 35 moles of ethylene oxide per mole of alcohol, and more especially the C₁₀-C₁₅ primary and secondary aliphatic alcohols ethoxylated with an average of from 1 to 10 moles of ethylene oxide per mole of alcohol.

### Zwitterionic Surfactants

In at least one embodiment, the formulation comprises a zwitterionic surfactant. The liquid laundry detergent formulation may comprise a zwitterionic surfactant, e.g. amine oxide or betaine, preferably in an amount of up to 10 wt.-% based on the total weight of the liquid laundry detergent formulation. Betaines may be alkyldimethyl betaines or alkylamido betaines, wherein the alkyl groups have C₁₂-C₁₈ chains.

### Additional Surfactants

In at least one embodiment, the liquid laundry detergent formulation comprises a surfactant selected from the group consisting of anionic surfactants, nonionic surfactants, and mixtures thereof; preferably the surfactant is selected from the group consisting of linear alkyl benzene sulfonates, alkyl ether sulfates, nonionic surfactants, amine oxides and betaines; and more preferably selected from the group consisting of linear alkyl benzene sulfonates, alkyl ether sulfates and nonionic surfactants. Other surfactants than the preferred LAS, AES, and nonionic surfactants may also be added. Although less preferred, alkyl sulfate surfactants may be used, especially the non-ethoxylated C₁₂-C₁₅ primary and secondary alkyl sulfates. Soap may also be used. Levels of soap are preferably lower than 10 wt.-%.

Preferably, the one or more surfactants in the liquid laundry detergent formulations are present in an amount of at least 5 wt.-%, preferably from 5 wt.-% to 65 wt.-%, more preferably from 6 wt.-% to 60 wt.-% and particularly preferably from 7 wt.-% to 55 wt.-%, in each case based on the total weight of the liquid laundry detergent formulation.

### Further Optional Ingredients

The household cleaning formulations may comprise one or more optional ingredients, e.g. they may comprise conventional ingredients commonly used in detergent compositions, especially laundry detergent compositions. Examples of optional ingredients include, but are not limited to builders, bleaching agents, bleach active compounds, bleach activators, bleach catalysts, photobleaches, dye transfer inhibitors, color protection agents, anti-redeposition agents, dispersing agents, fabric softening and antistatic agents, fluorescent whitening agents, enzymes, enzyme stabilizing agents, foam regulators, defoamers, malodor reducers, preservatives, disinfecting agents, hydrotropes, fibre lubricants, anti-shrinkage agents, buffers, fragrances, processing aids, colorants, dyes, pigments, anti-corrosion agents, fillers, stabilizers or other conventional ingredients for washing or laundry detergent compositions.

### Polymer

For detergency boosting, it may be advantageous to use a polymer in the household cleaning formulations, particularly in the liquid laundry detergent formulations. This polymer is preferably a polyalkoxylated polyethyleneimine (EPEI). Polyethylene imines are materials composed of ethylene imine units —CH₂CH₂NH— and, where branched, the hydrogen on the nitrogen is replaced by another chain of ethylene imine units. These polyethyleneimines can be prepared, for example, by polymerizing ethyleneimine in the presence of a catalyst such as carbon dioxide, sodium bisulfite, sulfuric acid, hydrogen peroxide, hydrochloric acid, acetic acid, or the like. Specific methods for preparing these polyamine backbones are disclosed in U.S. Pat. Nos. 2,182,306, 3,033,746, 2,208,095, 2,806,839, and 2,553,696.

The household cleaning formulations, particularly the liquid laundry detergent formulations, may comprise other polymeric materials, for example: dye transfer inhibition polymers, anti-redeposition polymers or cotton soil release polymers, especially those based on modified cellulosic materials. Especially, when EPEI is not present, the formulation may further comprise a polymer of polyethylene glycol and vinyl acetate, for example the lightly grafted copolymers described in WO 2007/138054. Such amphiphilic graft polymers based on water soluble polyalkylene oxides as graft base and side chains formed by polymerization of a vinyl ester component have the ability to enable reduction of surfactant levels whilst maintaining high levels of oily soil removal.

### Hydrotropes

In at least one embodiment, the formulation comprises a hydrotrope. Herein “hydrotrope” is a solvent that is neither water nor conventional surfactant, and that aids the solubilisation of surfactants and other components, especially any polymer and/or sequestrant, in the liquid, to render it isotropic. Hydrotropes are particularly useful in household cleaning formulations. Among suitable hydrotropes the following are noteworthy: monopropylene glycol (MPG), glycerol, sodium cumene sulfonate, ethanol, other glycols, e.g. dipropylene glycol, diethers and urea. MPG and glycerol are preferred hydrotropes.

### Enzymes

In at least one embodiment, the formulation, particularly the liquid laundry detergent formulation, comprises an enzyme. In at least one embodiment, the enyzme is selected from the group consisting of protease, mannanase, pectate lyase, cutinase, esterase, lipase, amylase, cellulase, and combinations thereof. Less preferred additional enzymes may be selected from peroxidase and oxidase. The enzymes are preferably present with corresponding enzyme stabilizers. The total enzyme content in the formulation is preferably from 0 wt. % to 5 wt.-%, more preferably from 0.5 wt.-% to 5 wt.-%, even more preferably from 1 wt.-% to 4 wt.-%, by total weight of the formulation.

### Sequestrants

Sequestrants are preferably included in the formulation, particularly in the household cleaning formulations. Preferred sequestrants include organic phosphonates, alkanehydroxy phosphonates and carboxylates, for example available under the DEQUEST trade mark from Thermphos. The preferred sequestrant level is less than 10 wt.-% and preferably less than 5 wt.-% by total weight of the formulation. A particularly preferred sequestrant is HEDP (1-hydroxyethylidene-1,1-diphosphonic acid), for example sold as Dequest 2010. Also suitable is Dequest® 2066 (diethylenetriamine penta(methylene-phosphonic acid) or Heptasodium DTPMP).

### Buffers

In at least one embodiment, the formulation, particularly the liquid laundry detergent formulation, comprises a buffer. In addition to agents optionally included for the generation of anionic surfactants, e.g. from LAS or fatty acids, the presence of buffer is preferred for pH control. Possible buffers are one or more ethanolamines, e.g. monoethanolamine (MEA) or triethanolamine (TEA). They are preferably used in formulation at levels of from 1.0 wt.-% to 15 wt.-%. Other suitable amino alcohol buffer materials may be selected from the group consisting of compounds having a molecular weight above 61 g/mol, which includes MEA. Suitable materials also include, in addition to the already mentioned materials: monoisopropanolamine, diisopropanolamine, triisopropanolamine, monoamino hexanol, 2-[(2-methoxyethyl)methylamino]-ethanol, propanolamine, N-methylethanolamine, diethanolamine, monobutanolamine, isobutanolamine, monopentanolamine, 1-amino-3-(2-methoxyethoxy)-2-propanol, 2-methyl-4-(methylamino)-2-butanol, or mixtures thereof. Potential alternatives to amino ethanol buffers are alkali hydroxides such as sodium hydroxide or potassium hydroxide.

The following examples are intended to further illustrate the invention without limiting the scope thereof.

### EXAMPLES

### Example 1—Sulfate-Free Liquid Handsoap (Amounts Indicated in wt-%)

| Example | 1a | 1b |
|---|---|---|
| Bio-based Sodium Cocoyl Isethionate | 1.18 | — |
| Bio-based Sodium Lauroyl Isethionate | — | 1.18 |
| Cocamidopropyl Betaine | 12.90 | 12.90 |
| Capryloyl/Caproyl Methyl | 3.80 | 3.80 |
| Glucamide (and) | | |
| Lauroyl/Myristoyl Methyl Glucamide | | |
| Glycerin | 7.00 | 7.00 |
| Sodium Benzoate | 0.50 | 0.50 |
| Trisodium Citrate | 0.50 | 0.50 |
| Citric Acid | q.s. to pH 4.5 | q.s. to pH 4.5 |
| PEG-150 Polyglyceryl-2 | 1.26 | 1.26 |
| Tristearate (and) | | |
| Laureth-3 (and) Glycol Distilled | | |
| Perfume | 0.3 | 0.3 |
| Dyestuff solution | q.s. | q.s. |
| Preservative | q.s. | q.s. |
| Water ad 100 | ad 100 | ad 100 |
| Total (%) | 100 | 100 |

### Example 2—Facial Cleansing Foam (Amounts Indicated in wt-%)

| Ingredient | Amount |
|---|---|
| Bio-based Sodium Isethionate | 2.00 |
| Stearic Acid | 8.20 |
| Myristic Acid | 0.50 |
| Lauric Acid | 7.00 |
| Palmitic Acid | 3.40 |
| Glycerin | 10.00 |
| PEG-8 | 10.00 |
| PEG-800 | 0.70 |
| Polyquaternium-43 | 0.70 |
| Sodium Cocoyl Glutamate | 2.00 |
| 1,3-Butandiol | 4.00 |
| Potassium Hydroxide | 3.60 |
| Perfume | 0.30 |
| Dyestuff solution | q.s. |
| Alcohol denat. | 2.00 |
| Tocopheryl Acetate | 0.50 |
| Water ad 100 | ad 100 |
| Total (%) | 100 |

### Example 3—Combo Bar Soap (Amounts Indicated in wt-%)

| Example | 3a | 3b |
|---|---|---|
| Bio-based Sodium Cocoyl Isethionate | 49.0 | — |
| Bio-based Sodium Lauroyl Isethionate | — | 49.0 |
| Stearic Acid | 18.00 | 18.00 |
| Sodium Tallowate | 10.00 | 10.00 |
| Bio-based Sodium Isethionate | 4.00 | 4.00 |
| Cococnut Fatty Acid | 5.00 | 5.00 |
| Cocamidopropyl Betaine | 3.00 | 3.00 |
| Sodium Chloride | 1.00 | 1.00 |
| Water ad 100 | ad 100 | ad 100 |
| Total (%) | 100 | 100 |

### Example 4—Sulfate-Free and Sodium Chloride-Free Shampoo (Amounts Indicated in wt-%)

| Example | 4a | 4b |
|---|---|---|
| Bio-based Sodium Cocoyl Isethionate | 2.30 | — |
| Bio-based Sodium Lauroyl Isethionate | — | 2.30 |
| Glycerin (85%) | 1.00 | 1.00 |
| Polyquaternium-10 | 0.30 | 0.30 |
| Hydroxypropyl Guar (and) Hydroxypropyl Guar | 0.15 | 0.15 |
| Hydroxypropyltrimon | | |
| Cocoyl Methyl Glucamide | 7.50 | 7.50 |
| Cocoamidopropyl Betaine | 16.67 | 16.67 |
| Sorbitan Caprylate, Propanediol, Benzoic Acid | 1.20 | 1.20 |
| Aqua, Glycol Distearate, Laureth-4, | 2.50 | 2.50 |
| Cocamidopropyl Betaine | | |
| Panthenol | 0.50 | 0.50 |
| Butylene Glycol, Water, Camellia Sinensis Leaf | 0.50 | 0.50 |
| Extract | | |
| Triethanolamine | q.s. | q.s. |
| Water ad 100 | ad 100 | ad 100 |
| Total (%) | 100 | 100 |

### Example 5—Solid Bar Shampoo (Amounts Indicated in wt-%)

| Example | 5a | 5b |
|---|---|---|
| Bio-based Sodium Cocoyl Isethionate | 20.0 | — |
| Bio-based Sodium Lauroyl Isethionate | — | 20.0 |
| Sodium Cocoyl Glycinate | 15.0 | 15.0 |
| Quaternium-98 | 4.0 | 4.0 |
| Cocamidopropyl Betaine | 10.0 | 10.0 |
| PEG-180 | 49.8 | 49.8 |
| Phenoxyethanol | 0.50 | 0.50 |
| Sorbitan Caprylate | 0.50 | 0.50 |
| Perfume | 0.2 | 0.2 |
| Water ad 100 | ad 100 | ad 100 |
| Total (%) | 100 | 100 |

### Example 6—Sulphate Free Shampoo (Amounts Indicated in wt-%)

| Example | 6a | 6b |
|---|---|---|
| Bio-based Sodium Cocoyl Isethionate | 5.0 | — |
| Bio-based Sodium Lauroyl Isethionate | — | 5.0 |
| Sodium C14-16 Olefin Sulfonate | 24.0 | 24.0 |
| Cocoyl Methyl Glucamide | 5.0 | 5.0 |
| Cocamidopropyl Betaine | 10.0 | 10.0 |
| Sorbitan Caprylate, Propanediol, Benzoic Acid | 0.8 | 0.8 |
| Polyquaternium-7 | 1.0 | 1.0 |
| PEG-150 Polyglyceryl-2 Tristearate (and) Laureth-3 | 1.0 | 1.0 |
| (and) Glycol Distilled | | |
| Sodium Chloride | 0.5 | 0.5 |
| Fragrance | q.s. | q.s. |
| Water ad 100 | ad 100 | ad 100 |
| Total (%) | 100 | 100 |

### Example 7—Bar Soap (Amounts Indicated in wt-%)

| Ingredient | Amount |
|---|---|
| Bio-based Sodium Cocoyl Isethionate, Stearic Acid | 64.5% |
| Lauroyl Sarcosine | 20.0% |
| Starch (Wheat) | 10.0% |
| Titanium Dioxide | 0.5% |
| Sodium Carbonate | 5.0% |

### Example 8—Bar Soap (Amounts Indicated in wt-%)

Example

| | 8a | 8b |
|---|---|---|
| Ingredient | Amount | Amount |
| Bio-based Sodium Cocoyl Isethionate | 5.0% | — |
| Bio-based Sodium Lauroyl Isethionate | — | 5.0% |
| Propylene glycol | 19.0% | 19.0% |
| Sorbitol | 12.0% | 12.0% |
| Sodium Laureth Sulfate | 17.8% | 17.8% |
| Alcohol denat. | 2.0% | 2.0% |
| Tocopheryl Acetate | 0.1% | 0.1% |
| Simmondsia Chinensis (Jojoba) Oil | 0.25% | 0.25% |
| Panthenol | 0.1% | 0.1% |
| Glycerin | 12.0% | 12.0% |
| Water | ad 100 | ad 100 |
| Stearic Acid | 13.0% | 13.0% |
| Myristic Acid | 6.0% | 6.0% |
| Sodium Hydroxide | 6.0% | 6.0% |
| Tetrasodium EDTA | 0.4% | 0.4% |
| Sodium Laureth-13 Carboxylate | 3.5% | 3.5% |
| Dyestuff solution | q.s. | q.s. |
| Fragrance | q.s. | q.s. |

### Example 9—Personal Care Formulation (Amounts Indicated in wt-%)

| | Ingredient | Amount |
|---|---|---|
| A | Bio-based Sodium Cocoyl Isethionate and stearic acid | 4.0% |
| B | Water | 53.9% |
| C | Genapol ® LRO liquid (Sodium Laureth Sulfate) | 30.0% |
| | Fragrance | 0.3% |
| | Genapol ® PDB [Glycoldistearate (and) Laureth-4 (and) | 4.0% |
| | Cocamidopropyl Betaine] | |
| | Genagen ® CAB (Cocamidopropyl Betaine) | 6.0% |
| | Dye solution | q.s. |
| | Preservative | q.s. |
| D | Sodium chloride | 1.8% |

Procedure: (I) Dissolve A in B while heating to 60° C., then cool down; (II) At approx. 35° C., add the components of C to I; (III) Adjust the pH if necessary; (IV) Adjust the viscosity with D

### Example 10—Shower Gel (Amounts Indicated in wt-%)

| | Ingredient | Amount |
|---|---|---|
| A | Allantoin | 0.40% |
| | Bio-based Sodium Cocoyl Isethionate and stearic acid | 4.0% |
| B | Water | 51.5% |
| C | Genapol ® LRO liquid (Sodium Laureth Sulfate) | 30.0% |
| | PEG-7 Glyceryl Cocoate | 2.0% |
| | Fragrance | 0.5% |
| | Genapol ® PGM (Sodium Laureth Sulfate, Glycol | 4.0% |
| | Distearate, Cocamide MEA) | |
| | Dye solution | q.s. |
| | Preservative | q.s. |
| | Genagen ® CAB (Cocamidopropyl Betaine) | 5.0% |
| | Genapol ® 26-L-3 (Genapol ® L-3) (Laureth-3) | 2.0% |
| D | Sodium chloride | 0.6% |

Procedure: (I) Dissolve the components of A in B while heating to approximately 60° C., then let cool down; (II) At approx. 35° C., add the components of C to I with stirring; (III) Adjust the pH if necessary; (IV) Adjust the viscosity with D

### Example 11—Household Cleaning Formulation (Hand Dishwashing Formulation) (Amounts Indicated in wt-%)

| | Example | | | | |
|---|---|---|---|---|---|
| | 11a | 11b | 11c | 11d | 11e |
| Bio-based Sodium | 18 | 18 | 19 | 8 | 18 |
| Lauroyl Isethionate | | | | | |
| Cocamidopropylbetaine | 8 | — | — | 4 | 4 |
| Sodium Alkane Sulfonate | — | — | — | 16 | — |
| Lauryldimethylamine | — | — | 6 | — | 4 |
| oxide | | | | | |
| Ethanol | 7 | 4 | — | — | — |
| Coco-Glucamide | — | 8 | — | — | — |
| Perfume | 0.2 | 0.2 | 0.1 | 0.1 | 0.1 |
| Cumene Sulfonate | — | — | 1.5 | — | — |
| Water & Auxiliaries | QSP | QSP | QSP | QSP | QSP |
| Total | 100% | 100% | 100% | 100% | 100% |

### Example 12—Household Cleaning Formulation (Hand Dishwashing Formulation) (Amounts Indicated in wt-%)

| | Example | | | | |
|---|---|---|---|---|---|
| | 12a | 12b | 12c | 12d | 12e |
| Bio-based Sodium | 18 | 18 | 18 | 18 | 18 |
| Lauroyl Isethionate | | | | | |
| Lauryldimethylamine | 4 | 8 | 4 | — | — |
| Oxide | | | | | |
| Laureth-7 | — | 2 | — | — | — |
| Cocamidopropylbetaine | — | — | — | 4 | 4 |
| Coco-Glucamide | | | 4 | 4 | — |
| Cocoyl Polyglykosid | 4 | — | — | — | 4 |
| Perfume | 0.2 | 0.2 | 0.1 | 0.1 | 0.8 |
| Water & Auxiliaries | QSP | QSP | QSP | QSP | QSP |
| Total | 100% | 100% | 100% | 100% | 100% |

### Example 13—Household Cleaning Formulation (Hard Surface Cleaner) (Amounts Indicated in wt-%)

| | Example | | | | |
|---|---|---|---|---|---|
| | 13a | 13b | 13c | 13d | 13e |
| Bio-based Sodium | 3 | 3 | 3 | 3 | 3 |
| Lauroyl Isethionate | | | | | |
| Lauryldimethylamine | 2 | — | — | — | 1 |
| Oxide | | | | | |
| Laureth-7 | — | 2 | — | — | — |
| Capryl/Caproyl-Glucamide | | | 2 | — | 1 |
| Capryl/Caproyl- | — | — | — | 2 | — |
| Polyglykosid | | | | | |
| Perfume | 0.2 | 0.2 | 0.1 | 0.1 | 0.2 |
| Water & Auxiliaries | QSP | QSP | QSP | QSP | QSP |
| Total | 100% | 100% | 100% | 100% | 100% |

### Example 14—Bar Soap (Amounts Indicated in wt-%)

Example

| | 14a | 14b |
|---|---|---|
| Ingredient | Amount | Amount |
| Bio-based Sodium Cocoyl Isethionate | 5.0% | — |
| Bio-based Sodium Lauroyl Isethionate | — | 5.0% |
| Propylene glycol | 20.0% | 20.0% |
| Sorbitol | 15.0% | 15.0% |
| Sodium Laureth Sulfate | 18.0% | 18.0% |
| Vitamin E | 0.1% | 0.1% |
| Simmondsia Chinensis (Jojoba) Oil | 0.25% | 0.25% |
| Glycerin | 14.0% | 14.0% |
| Water | 2.25% | 2.25% |
| Stearic Acid | 13.0% | 13.0% |
| Myristic Acid | 6.0% | 6.0% |
| Sodium Hydroxide (50% solution) | 6.0% | 6.0% |
| Disodium EDTA | 0.4% | 0.4% |
| Dyestuff solution | q.s. | q.s. |
| Fragrance | q.s. | q.s. |

1-15. (canceled)

16. A compound of Formula (I) 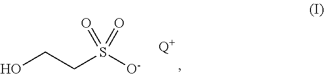
wherein
Q⁺ is a cation; and
the compound of Formula (I) has at least 50 wt-% bio-based carbon content, relative to the total mass of carbon in the compound.

17. The compound of Formula (I) according to claim 16, wherein the compound is derived from ethylene oxide having at least 50 wt-% bio-based carbon content, relative to the total mass of carbon in the ethylene oxide.

18. The compound of Formula (I) according to claim 17, wherein the ethylene oxide has at least 80 wt-% bio-based carbon content, relative to the total mass of carbon in the ethylene oxide.

19. The compound of Formula (I) according to claim 16, wherein Q⁺ is selected from H⁺, NH₄⁺, Li⁺, Na⁺, K⁺, ½ Ca⁺⁺, ½ Mg⁺⁺, ½ Zn⁺⁺, ⅓ Al⁺⁺⁺, and combinations thereof, preferably wherein Q⁺ is Na⁺.

20. The compound of Formula (I) according to claim 19, wherein Q⁺ is Na⁺.

21. The compound of Formula (I) according to claim 16, wherein the compound is sodium isethionate.

22. A compound of Formula (II) 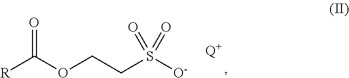
wherein
Q⁺ is a cation;
R is selected from saturated hydrocarbon chains having 5 to 23 carbon atoms, unsaturated hydrocarbon chains having 5 to 23 carbon atoms, and combinations thereof, or the R—C═O residue in Formula (II) is derived from coconut fatty acids; and
the compound of Formula (II) has at least 50 wt-% bio-based carbon content, relative to the total mass of carbon in the compound.

23. The compound of Formula (II) according to claim 22, wherein the compound is derived from ethylene oxide having at least 50 wt-% bio-based carbon content, relative to the total mass of carbon in the ethylene oxide.

24. The compound of Formula (II) according to claim 23, wherein the ethylene oxide has at least 80 wt-% bio-based carbon content, relative to the total mass of carbon in the ethylene oxide.

25. The compound of Formula (II) according to claim 22, wherein Q⁺ is selected from H⁺, NH₄⁺, Li⁺, Na⁺, K⁺, ½ Ca⁺⁺, ½ Mg⁺⁺, ½ Zn⁺⁺, ⅓ Al⁺⁺⁺, and combinations thereof.

26. The compound of Formula (II) according to claim 25, wherein Q⁺ is Na⁺.

27. The compound of Formula (II) according to claim 22, wherein R is selected from saturated hydrocarbon chains having 7 to 17 carbon atoms, or the R—C═O residue in Formula (II) is derived from coconut fatty acids.

28. The compound of Formula (II) according to claim 22, wherein the compound is sodium lauroyl isethionate or sodium cocoyl isethionate.

29. A process for preparing a compound of Formula (I) 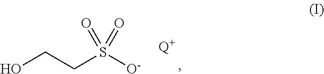
wherein
Q⁺ is a cation; and
the compound of Formula (I) has at least 50 wt-% bio-based carbon content, relative to the total mass of carbon in the compound,
the process comprising reacting ethylene oxide with a bisulfite, wherein the ethylene oxide has at least 50 wt-%, relative to the total mass of carbon in the ethylene oxide.

30. A process for preparing a compound of Formula (II) 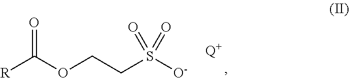
wherein
Q⁺ is a cation;
R is selected from saturated hydrocarbon chains having 5 to 23 carbon atoms, unsaturated hydrocarbon chains having 5 to 23 carbon atoms, and combinations thereof, or the R—C═O residue in Formula (II) is derived from coconut fatty acids; and
the compound of Formula (II) has at least 50 wt-% bio-based carbon content, relative to the total mass of carbon in the compound,
the process comprising reacting a compound of Formula (I) 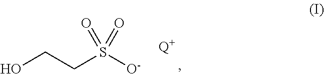
wherein
Q⁺ is a cation; and
the compound of Formula (I) has at least 50 wt % bio-based carbon content, relative to the total mass of carbon in the compound,
with a compound of Formula (III)
RC(═O)OH (III),
wherein R is as defined in Formula (II).

31. A formulation comprising
(a) from 0.1 to 90 wt-% of a compound of Formula (I) as defined in claim 16; and
(b) from 10 to 99.9 wt-% of one or more further components.

32. A formulation comprising
(a) from 0.1 to 90 wt-% of a compound of Formula (II) as defined in claim 22; and
(b) from 10 to 99.9 wt-% of one or more further components.

33. The formulation according to claim 31, wherein the formulation is a household cleaning formulation.

34. The formulation according to claim 32, wherein the formulation is a household cleaning formulation.

35. A surfactant consisting of the compound of Formula (II) according to claim 22.
